(19) United States
(12) Patent Application Publication
(10) Pub. No.: US 2014/0251321 A1
(43) Pub. Date: Sep. 11, 2014

(54) INHALER MOUTHPIECE

(71) Applicant: Jonah Henry Benson, West Bloomfield, MI (US)

(72) Inventor: Jonah Henry Benson, West Bloomfield, MI (US)

(21) Appl. No.: 13/789,561

(22) Filed: Mar. 7, 2013

(51) Int. Cl. A61M 15/00

(52) U.S. Cl. CPC A61M 15/009
USPC 128/200.23

(57) ABSTRACT

A detachable mouthpiece for an inhaler apparatus constructed to convey an aerosolized substance into a user's lungs via the user's mouth. The detachable mouthpiece includes a detachable mouthpiece body having a first distal end defining a first opening and a second distal end. The detachable mouthpiece also includes a feature positioned at the second distal end of the detachable mouthpiece body. The feature is configured to selectively attach the detachable mouthpiece at the second distal end to and detach the detachable mouthpiece from a housing of the inhaler apparatus. The first opening is configured to convey the aerosolized substance from the inhaler apparatus to the user's mouth when the detachable mouthpiece is secured to the housing of the inhaler apparatus. An inhaler apparatus having such a detachable mouthpiece is also disclosed.

### CROSS-REFERENCE TO RELATED APPLICATIONS

This application claims the benefit of United States Provisional Patent Application No. 61/673,704 filed on Jul. 19, 2012, which is hereby incorporated by reference in its entirety.

### TECHNICAL FIELD

The present disclosure relates to a mouthpiece for an inhaler of the type used to administer a medication into a patient's lungs.

### BACKGROUND

An inhaler is a device used for delivering a medication into the body of a patient or user of the device via the user's lungs. Inhalers are frequently used in the treatment of asthma, Chronic Obstructive Pulmonary Disease (COPD), and other respiratory diseases. For example, a medication such as Znamivir (Relenza), which is used to treat influenza, is administered via an inhaler. Although various types of inhalers, such as dry-powder and nebulizer devices, have been employed for administering medications, a metered-dose inhaler (MDI) is the most common type used today.

The MDI is designed for delivering a specific amount of aerosolized medication, typically in the form of a short burst, to the lungs of the user. In an MDI, medication is most commonly stored in a canister containing a propellant. The propellant is pressurized to provide a force for generating an aerosol cloud and is also the medium in which the medication is suspended or dissolved.

The MDI may include a complementary tube or spacer designed to reduce deposition of the medication in the user's mouth and throat and reduce the need for precise synchronization of the start of inhalation with the actuation of the MDI. The spacer is generally positioned between the canister and the user's mouth, and functions as a holding chamber for the medication in aerosolized form. Spacers can be especially helpful to individuals who find a regular MDI sans spacer difficult to operate.

Regardless of the inhaler type, generally the subject inhaler also includes a mouthpiece that provides an interface between the inhaler and the user's mouth. In an MDI the mouthpiece may be incorporated into the spacer or directly into the canister structure.

### SUMMARY

A detachable mouthpiece is disclosed for an inhaler apparatus constructed to dispense an aerosolized substance into a user's lungs via the user's mouth. The detachable mouthpiece may function as an auxiliary mouthpiece for an inhaler apparatus that already includes a primary mouthpiece. The detachable mouthpiece includes a detachable mouthpiece body having a first distal end and a second distal end, wherein the first distal end defines a first opening. The detachable mouthpiece also includes a feature positioned at the second distal end of the detachable mouthpiece body. The feature is configured to selectively secure the detachable mouthpiece at the second distal end and disconnect the detachable mouthpiece from the inhaler apparatus. The first opening is configured to convey the aerosolized substance from the inhaler apparatus to the user's mouth when the detachable mouthpiece is secured or attached to the inhaler apparatus.

An inhaler apparatus having such a detachable mouthpiece is also disclosed. Such an inhaler apparatus may be configured as a metered-dose inhaler (MDI).

The inhaler apparatus may include an inhaler apparatus housing having a primary mouthpiece. The inhaler apparatus may include a canister configured to hold the first substance and engaged with the inhaler apparatus housing such that the primary mouthpiece is in fluid communication with the canister. In such a case, the detachable mouthpiece may be configured for selective attachment to and detachment from the primary mouthpiece.

Additionally, the primary mouthpiece may define a discharge nozzle configured to convey the aerosolized first substance from the canister into the user's mouth when only the primary mouthpiece is used, i.e., when the detachable mouthpiece is disconnected from the primary mouthpiece. On the other hand, when the detachable mouthpiece is attached to the primary mouthpiece, the detachable mouthpiece may receive the aerosolized first substance from the canister via the discharge nozzle, thus allowing the first opening to convey the aerosolized first substance to the user's mouth.

The feature may be configured to generate a tight or interference-type fit between the detachable mouthpiece and the primary mouthpiece for securing the detachable mouthpiece to the primary mouthpiece.

The detachable mouthpiece and the feature may be formed from dissimilar materials and combined into a unitary, interconnected structure. In such a case, the material of the feature may be pliant, while the material of the detachable mouthpiece body may be substantially rigid.

The feature may be configured as a projection or a ridge.

The detachable mouthpiece may be a monolithic structure formed in its entirety from a single pliant material.

The first substance may include a medication that is either suspended or dissolved in a propellant, wherein the medication is characterized by at least one of a distasteful scent and flavor. Additionally, the detachable mouthpiece may be impregnated with a second substance. In such a case, the second substance may be characterized by at least one of a pleasing scent/aroma and flavor. The pleasing scent/aroma and flavor of the second substance may be configured to counteract, i.e., neutralize/offset or mask/cover, the at least one of the distasteful scent and flavor, respectively, of the first substance.

The detachable mouthpiece may include a portion characterized by a distinct, vivid, or vibrant tint.

The portion may be configured to glow, i.e., radiate light, in low light conditions.

The above features and advantages, and other features and advantages of the present disclosure, will be readily apparent from the following detailed description of the embodiment(s) and best mode(s) for carrying out the described invention when taken in connection with the accompanying drawings and appended claims.

### BRIEF DESCRIPTION OF THE DRAWINGS

FIG. 1 is a schematic side view of a metered-dose inhaler (MDI) apparatus having a primary mouthpiece and a detachable mouthpiece configured as an auxiliary mouthpiece.

FIG. 2 is a schematic partial cross-sectional side view of an inhaler apparatus shown in FIG. 1, but without the primary mouthpiece and the detachable mouthpiece shown according to one embodiment.

FIG. 3 is a schematic partial cross-sectional side view of an inhaler apparatus similar to the apparatus shown in FIG. 2, but with the detachable mouthpiece shown according to another embodiment.

FIG. 4 is a schematic side view of the detachable mouthpiece shown in FIGS. 1 and 2.

FIG. 5 is a schematic side view of the detachable mouthpiece shown in FIG. 3.

FIG. 6 is a schematic side view of a nebulizer-type inhaler apparatus employing the detachable mouthpiece as shown in FIGS. 2-5.

### DETAILED DESCRIPTION

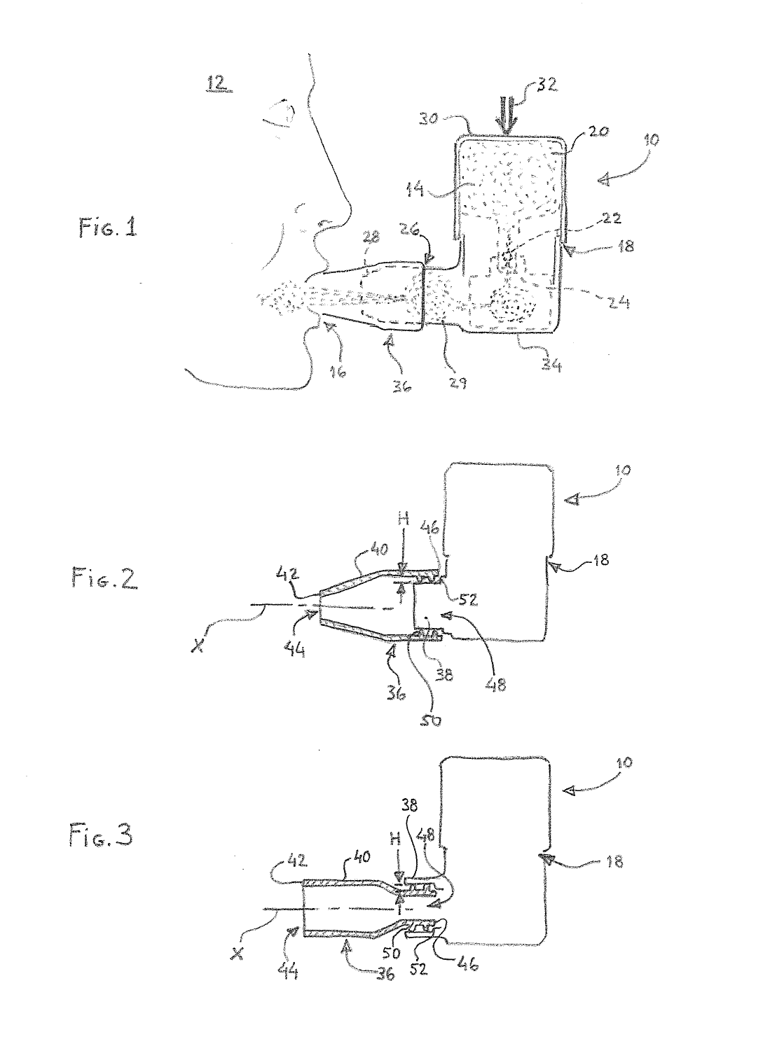

Referring to the drawings, wherein like reference numbers refer to like components, FIG. 1 shows a side view of an inhaler apparatus 10. The inhaler apparatus 10 is employed for delivering a medication into the lungs of a patient or user 12. The inhaler apparatus 10 may be a metered-dose inhaler (MDI), as shown in FIG. 1 that is configured to dispense a predetermined dose or quantity of an aerosolized first substance 14 into the user's lungs via the user's mouth 16.

As shown in FIG. 1, the inhaler apparatus 10 includes an inhaler housing 18. The inhaler housing 18 may be constructed from any appropriate material that is not chemically reactive with the first substance 14, such as moldable plastic. The inhaler apparatus 10 also includes a canister 20 engaged with the inhaler housing 18. The canister 20 holds the first substance 14 and includes a metering valve 22 configured to establish the predetermined quantity of the first substance 14 to be aerosolized and dispensed with each actuation of the inhaler apparatus 10. The first substance 14 is pressurized inside the canister 20 and thus facilitates closing of the metering valve 22 following each actuation of the inhaler apparatus 10 by the user 12.

The canister 20 is inserted into a receptacle or orifice 24 that is defined by the structure of the inhaler housing 18 and configured to direct the flow of the first substance 14 toward a specifically contoured mouthpiece 26. As shown, the mouthpiece 26 is in fluid communication with the canister 20 and configured to convey the aerosolized first substance 14 into the user's lungs. Although, the mouthpiece 26 is shown in FIG. 1, the subject mouthpiece may be omitted from the inhaler apparatus 10. When included in the inhaler apparatus 10, the mouthpiece 26 functions as a primary mouthpiece adapted to convey the first substance 14 into the user's mouth 16.

As may be seen in FIG. 1, the mouthpiece 26 includes a discharge nozzle 28 for engagement with the user's mouth 16. The inhaler apparatus 10 may also include a dust cap (not shown) configured to fit over the discharge nozzle 28 in order to prevent contamination of the discharge nozzle while the inhaler apparatus 10 is not in use. The mouthpiece 26 may include a spacer or holding chamber 29 disposed downstream of the canister 20 for holding the first substance 14 in aerosolized form prior to the first substance being discharged into the user's mouth 16. Although the inhaler apparatus 10 is depicted and described hereafter as an MDI, the present disclosure is equally applicable to other types of inhalers, for example dry-powder or nebulizer (shown in FIG. 6) devices that deliver a formulation such as the first substance 14 into the body of the user 12 via the user's mouth 16. In the MDI, the canister 20 may be a pressurized container for holding the first substance 14, while in other types of inhalers the canister may be a sealed but not pressurized container configured to hold the first substance until accessed via operation of the inhaler by the user.

As shown in FIG. 1, to operate the inhaler apparatus 10, the user 12 presses down on a top portion 30 of the canister 20 with a force 32, while the user's thumb supports a lower portion 34 of the inhaler housing 18. Actuation of the inhaler apparatus 10 typically releases a single metered dose of the first substance 14, which may include a medication and a stabilizing excipient that may be either dissolved or suspended in a volatile propellant. Breakup of such a propellant into droplets, followed by rapid evaporation of those droplets, results in the generation of an aerosolized first substance 14 consisting of micrometer-sized medication particles that may then be inhaled by the user 12. Consequently, in general the inhaler apparatus 10 holds the first substance 14 that is aerosolized for inhalation on demand from the user 12.

With continued reference to FIG. 1, the inhaler apparatus 10 also includes a detachable mouthpiece 36. The detachable mouthpiece 36 is configured, i.e., designed and constructed, for selective attachment to and detachment from the inhaler housing 18. In the absence of the primary mouthpiece 26, the detachable mouthpiece 36 is configured for direct attachment to a section 38 (shown in FIGS. 2 and 3) of the inhaler housing 18. On the other hand, in the event that the inhaler apparatus 10 includes the primary mouthpiece 26, the detachable mouthpiece 36 may function as an auxiliary mouthpiece and be configured for attachment to the primary mouthpiece. The detachable mouthpiece 36 includes a detachable mouthpiece body 40 having a first distal end 42 defining a first opening 44 and a second distal end 46 defining a second opening 48.

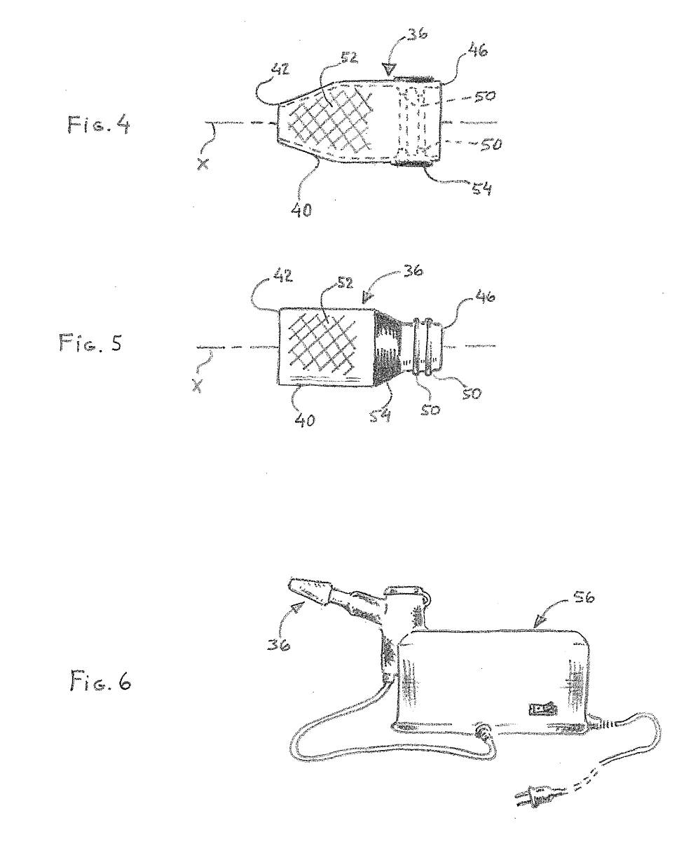

The first opening 44 is configured to convey the aerosolized first substance 14 from the inhaler apparatus 10 to the user's mouth 16 when the detachable mouthpiece 36 is attached, i.e., secured, to the inhaler apparatus 10. Specifically, the first opening 44 is configured for engagement with the user's mouth 16, while the second opening 48 is configured to mate up to the inhaler housing 18. In the absence of the primary mouthpiece 26, the first opening 44 discharges the first substance 14 into the user's mouth 16 after the second opening 48 receives the first substance directly from the canister 20 (as shown in FIG. 1). Conversely, when the detachable mouthpiece 36 is secured to the primary mouthpiece 26, the detachable mouthpiece 36 initially receives the first substance 14 from the canister 20 via the discharge nozzle 28, and then the first opening 44 conveys the first substance to the user's mouth 16 (as shown in FIGS. 3-4).

As may be seen in FIGS. 2-5, the detachable mouthpiece 36 includes a feature 50 that is positioned at the second distal end 48. The feature 50 is configured to selectively secure the detachable mouthpiece 36 at the second distal end to the inhaler apparatus housing 18 and enable expedient separation of the detachable mouthpiece from the housing 18. The feature 50 may be configured to generate a tight or interference-type fit between the detachable mouthpiece 36 and the primary mouthpiece 26 for securing the detachable mouthpiece to the primary mouthpiece. The detachable mouthpiece 36 may be characterized by a monolithic construction, i.e., formed as a uniform structure from a single material. Such a detachable mouthpiece 36, including the feature 50, may be shaped or formed from a single pliant material.

The inhaler apparatus 10 may additionally include a surface formation 52, such as a step (shown in FIGS. 2 and 3) or a channel (not shown). The surface formation 52 is configured to engage the feature 50 for a more positive attachment of the detachable mouthpiece 36 to the inhaler housing 18, such as by providing a definitive stop for the detachable mouthpiece relative to the inhaler housing. Accordingly, depending on whether the detachable mouthpiece 36 is intended to connect to the primary mouthpiece 26 or to the section 38, the surface formation 52 may be located on the primary mouthpiece or directly on the section (as shown).

Materials of the detachable mouthpiece body 40 and the feature 50 may be plastically moldable. The detachable mouthpiece body 40 and the feature 50 may also be formed or molded from dissimilar materials, as shown in FIG. 5. Furthermore, the detachable mouthpiece body 40 and the feature 50 may be combined into a single interconnected structure. In such a case, the material of the feature 50 may be pliant, while the material of the detachable mouthpiece body 40 may be substantially rigid. In the event that the material of the feature 50 is pliant, the feature will compress against the section 38 as the detachable mouthpiece 36 is pushed onto the inhaler housing 18. Additionally, as the detachable mouthpiece 36 is being pushed onto the inhaler housing 18, the rigid detachable mouthpiece body 40 may provide a suitably robust support for the feature 50.

The feature 50 may be a natural physical edge or surface of the distal end 48 that is configured to generate a tight fit with respect to the housing 18 or the primary mouthpiece 26. In the alternative, the feature 50 may be a projection or a ridge. Additionally, the feature 50 may be a series of such projections interspaced along the length of the detachable mouthpiece body 40 (as shown in FIGS. 2-5). Such a projection or series of projections may be concentric with respect to a center axis X of the detachable mouthpiece body 40. The feature 50 may substantially circumscribe the inner surface of the detachable mouthpiece body 40, and thus substantially circumscribe the inner surface, as shown in a cross-sectional view in FIG. 2 and in phantom lines in FIG. 4.

In an alternative embodiment, if the feature 50 is configured as a projection or a series of projections, the feature 50 may substantially circumscribe the outer surface of the detachable mouthpiece body 40, as shown in FIGS. 3 and 5. The actual shape of the feature 50 may be selected to complement the external shape of the mouthpiece 26. For example, as may be seen in FIGS. 2 and 3, the feature 50 includes a height H, which may be selected based on the durometer of the material, i.e., the measure of material's hardness, selected for the feature. Additionally, the durometer of material for the feature 50 may be selected based on the amount of compression generated by the push-on force required to secure the detachable mouthpiece 36 onto the inhaler housing 18. Furthermore, if the feature 50 is configured as a series of projections, such may have a graduated, or variable height H as deemed appropriate for secure engagement of the detachable mouthpiece 36 to the inhaler housing 18.

As described above, the first substance 14 may include a medication that is either suspended or dissolved in a propellant. Such medication may be characterized by a distasteful scent and/or flavor, which may make regular administering of the treatment problematic, especially to children. To address the foregoing concern, the detachable mouthpiece 36 may be impregnated or saturated with a second substance 52 such that the user 12 may sense the second substance during operation of the inhaler apparatus 10. The second substance 52 may be specifically formulated to counteract the distasteful scent and/or flavor of the first substance 14. To such an end, the second substance 52 may be characterized by a pleasing scent or aroma intended to offset, i.e., neutralize, and/or mask, i.e., cover, the distasteful scent of the first substance 14. Furthermore, the second substance 52 may be characterized by a pleasing flavor intended to offset and/or mask the distasteful flavor of the first substance 14.

As shown in FIGS. 4 and 5, the detachable mouthpiece 36 may include a portion 54 characterized by a distinct, vivid, and/or vibrant tint. The portion 54 may be a localized section on or extend continuously around the body 40. Furthermore, the portion 54 may be configured to be luminous, i.e., glow or radiate light, during low or reduced light conditions. Such luminous portion 54 may facilitate locating the detachable mouthpiece 36 in the dark, if the detachable mouthpiece resides alone, or locating the entire inhaler apparatus 10 when the detachable mouthpiece is connected to the inhaler housing 18 or to the primary mouthpiece 26. Additionally, the distinctly tinted portion 54 may also be preferred for its unique appearance. Alternately, the entire detachable mouthpiece 36 may be characterized by a distinct tint. The distinct tint of either the portion 54 or the entire detachable mouthpiece 36 may be used to distinguish one detachable mouthpiece from another detachable mouthpiece, thus facilitating usage of a single inhaler housing 18 with two distinct detachable mouthpieces, such as by two individual users.

The detachable mouthpiece 36 may be disconnected from the inhaler housing 18 for separate storage. After the detachable mouthpiece 36 is disconnected from the inhaler housing 18, the inhaler housing may be stored out in the open, while the detachable mouthpiece 36 may be stored in its own individual container, thus being covered from contamination. Furthermore, the detachable mouthpiece 36 may be a disposable item, thus allowing a new detachable mouthpiece to be employed every time the inhaler apparatus 10 is put to use. As shown in FIG. 6, other types of inhalers such as a nebulizer 56 may also employ the detachable mouthpiece 36 as disclosed in the foregoing description. The detachable mouthpiece 36 used on the nebulizer 56 may be of the type shown in FIGS. 2 and 4 or of the type shown in FIGS. 3 and 5.

The detailed description and the drawings or figures are supportive and descriptive of the invention, but the scope of the invention is defined solely by the claims. While some of the best modes and other embodiments for carrying out the claimed invention have been described in detail, various alternative designs and embodiments exist for practicing the invention defined in the appended claims.

1. An inhaler apparatus for dispensing an aerosolized first substance into a user's lungs via the user's mouth, the inhaler apparatus comprising:
an inhaler housing configured to hold the first substance that is aerosolized on demand from the user; and
a detachable mouthpiece configured to be selectively attached to and detached from the inhaler housing, the detachable mouthpiece having:
a detachable mouthpiece body having a first distal end defining a first opening and a second distal end; and
a feature positioned at the second distal end of the detachable mouthpiece body;
wherein:
the feature is configured to selectively attach the detachable mouthpiece at the second distal end to and detach the detachable mouthpiece from the inhaler housing; and
the first opening is configured to convey the aerosolized substance from the inhaler housing to the user's mouth when the detachable mouthpiece is attached to the inhaler housing.

2. The inhaler apparatus according to claim 1, further comprising a canister engaged with the inhaler housing and configured to hold the first substance;
wherein:
the inhaler housing includes a primary mouthpiece in fluid communication with the canister;
the detachable mouthpiece is configured for selective attachment to and detachment from the primary mouthpiece;
the primary mouthpiece defines a discharge nozzle configured to convey the aerosolized first substance from the canister into the user's mouth when the detachable mouthpiece is detached from the primary mouthpiece; and
the detachable mouthpiece receives the aerosolized first substance from the canister via the discharge nozzle and the first opening conveys the aerosolized first substance to the user's mouth when the detachable mouthpiece is attached to the primary mouthpiece.

3. The inhaler apparatus according to claim 2, wherein the feature is configured to generate a tight fit between the detachable mouthpiece and the primary mouthpiece for securing the detachable mouthpiece to the primary mouthpiece.

4. The inhaler apparatus according to claim 1, wherein the detachable mouthpiece and the feature are formed from dissimilar materials and combined into a unitary structure, and wherein the material of the feature is pliant and the material of the detachable mouthpiece body is substantially rigid.

5. The inhaler apparatus according to claim 1, wherein the feature is configured as a projection.

6. The inhaler apparatus according to claim 1, wherein the detachable mouthpiece body is a monolithic structure formed from a pliant material.

7. The inhaler apparatus according to claim 1, wherein:
the first substance includes a medication characterized by at least one of a distasteful scent and flavor;
the detachable mouthpiece is impregnated with a second substance; and
the second substance is characterized by at least one of a scent and flavor configured to counteract the at least one of the distasteful scent and flavor, respectively, of the first substance.

8. The inhaler apparatus according to claim 1, wherein the detachable mouthpiece includes a portion characterized by a distinct tint.

9. The inhaler apparatus according to claim 8, wherein the portion is configured to glow in low light conditions.

10. The inhaler apparatus according to claim 1, wherein the inhaler apparatus is a metered-dose inhaler.

11. A detachable mouthpiece for an inhaler apparatus constructed to dispense an aerosolized substance into a user's lungs via the user's mouth, the detachable mouthpiece comprising:
a detachable mouthpiece body having a first distal end defining a first opening and a second distal end; and
a feature positioned at the second distal end of the detachable mouthpiece body;
wherein:
the feature is configured to selectively attach the detachable mouthpiece at the second distal end to and detach the detachable mouthpiece from the inhaler apparatus; and
the first opening is configured to convey the aerosolized substance from the inhaler apparatus to the user's mouth when the detachable mouthpiece is attached to the inhaler apparatus.

12. The detachable mouthpiece according to claim 11, wherein:
the inhaler apparatus includes an inhaler apparatus housing having a primary mouthpiece, a canister configured to hold the first substance and engaged with the inhaler apparatus housing, wherein the primary mouthpiece is in fluid communication with the canister;
the detachable mouthpiece is configured as an auxiliary mouthpiece for selective attachment to and detachment from the primary mouthpiece;
the primary mouthpiece defines a discharge nozzle configured to convey the aerosolized first substance from the canister into the user's mouth when the detachable mouthpiece is detached from the primary mouthpiece; and
the detachable mouthpiece receives the aerosolized first substance from the canister via the discharge nozzle and the first opening conveys the aerosolized first substance to the user's mouth when the detachable mouthpiece is attached to the primary mouthpiece.

13. The detachable mouthpiece according to claim 12, wherein the feature is configured to generate a tight fit between the detachable mouthpiece and the primary mouthpiece for securing the detachable mouthpiece to the primary mouthpiece.

14. The detachable mouthpiece according to claim 11, wherein the detachable mouthpiece and the feature are formed from dissimilar materials and combined into a unitary interconnected structure, and wherein the material of the feature is pliant and the material of the detachable mouthpiece body is substantially rigid.

15. The detachable mouthpiece according to claim 11, wherein the feature is configured as a projection.

16. The detachable mouthpiece according to claim 11, wherein the detachable mouthpiece is a monolithic structure formed from a pliant material.

17. The detachable mouthpiece according to claim 11, wherein the detachable mouthpiece is impregnated with a second substance.

18. The detachable mouthpiece according to claim 17, wherein:
the first substance includes a medication characterized by at least one of a distasteful scent and flavor; and
the second substance is characterized by at least one of a scent and flavor configured to counteract the at least one of the distasteful scent and flavor, respectively, of the first substance.

19. The detachable mouthpiece according to claim 11, wherein the detachable mouthpiece includes a portion characterized by a distinct tint.

20. The detachable mouthpiece according to claim 19, wherein the portion is configured to glow in low light conditions.
